(19) United States
(12) Patent Application Publication
(10) Pub. No.: US 2010/0136545 A1
(43) Pub. Date: Jun. 3, 2010

(54) Cytokines and Genes Differentially Affected by TNF Blockers

(75) Inventor: Robert Wallis, Washington, DC (US)

Correspondence Address: PATENT DOCKET ADMINISTRATOR;LOWENSTEIN SANDLER PC, 65 LIVINGSTON AVENUE, ROSELAND, NJ, 07068, US

(73) Assignee: UNIVERSITY OF MEDICINE AND DENTISTRY OF NEW JERSEY, Somerset, NJ (US)

(21) Appl. No.: 12/531,640

(22) PCT Filed: Mar. 14, 2008
(86) PCT No.: PCT/US08/57052

§ 371 Date: Jan. 29, 2010

Related U.S. Application Data
(60) Provisional application No. 60/918,732, filed on Mar. 19, 2007.

(51) Int. Cl. C12Q 1/68; G01N 33/53

(52) U.S. Cl. 435/6; 435/7.92

(57) ABSTRACT

The present invention is directed to cytokines and genes that are differentially affected by TNF blockers and the use of these genes and cytokines to help asses the TB risks of new immunosuppressive therapies, to help evaluate the effects of new TB vaccines, and to help assess TB susceptibility in persons exposed to Mycobacterium tuberculosis.

### RELATED APPLICATIONS

This application claims priority to Provisional Application No. 60/918,732, filed Mar. 19, 2007, the disclosure of which is hereby incorporated h reference in its entirety.

### STATEMENT REGARDING REFERENCES

All patents, publications and non-patent references referred to herein shall be considered incorporated by reference into this application in their entireties.

### BACKGROUND OF THE INVENTION

Tumor necrosis factor (TNF) plays a key role as a cause of many chronic inflammatory diseases such as rheumatoid arthritis (RA), but is essential for host defenses against many infectious such as tuberculosis (TB). The most widely used TNF blockers differ substantially in their risk of reactivating latent TB infection, with infliximab (Remicade, a monoclonal antibody) being many times that of etanercept (Enbrel, soluble TNF receptor).

Recognition of the central role of TNF in the pathogenesis of chronic inflammatory disease such as RA has profoundly changed the treatment of these conditions, with the introduction into clinical use of infliximab in 1998, etanercept in 1999, and adalimumab in 2004. As experience with these drugs has grown, important differences between them have emerged. Despite sharing a common therapeutic target, infliximab poses a 9 times greater risk of reactivation of latent M. tuberculosis infection during the first 3 months of treatment than etanercept. (See, e.g., Wallis et al, Clin Infect. Dis 2005). Histoplasmosis, listeriosis, and coccidioidomycosis also are increased with infliximab. (See, e.g., Bergstrom et al. Arthritis Rheum 2004; and Lee et al. Arthritis Rheum 2002).

Thus, there remains a need in the art provide a tool for understanding the different effects TNF may have on cells in order to evaluate current and future treatments, assess the TB risks of new immunosuppressive therapies, evaluate the effects of new TB vaccines, and assess TB susceptibility in persons exposed to Mycobacterium tuberculosis.

The model of the present invention distinguishes cytokines and genes that are differentially affected by TNF blockers, which can then be used to address these issues.

### BRIEF DESCRIPTION OF THE FIGURES

FIG. 1 depicts the effect of TNF blockade on antigen-induced production of IFNγ (FIG. 1(a)) and IL-10 (FIG. 1(b)) in whole blood cultures stimulated with M. tuberculosis CF. Symbols represent median and interquartile range (IQR) of 15 subjects. Median IFNγ and IL-10 levels without TNF blockers were 1028 and 875 pg/ml, respectively. Asterisks indicate statistical significance compared to untreated control cultures by repeated measures ANOVA on ranks followed by post-hoc testing using Dunn's method. Both trough and peak concentrations of adalimumab and infliximab inhibited IFNγ production, whereas etanercept did not, even at a supratherapeutic concentration. All three drugs inhibited production of IL-10 at all concentrations tested.

FIG. 2 depicts the effect of TNF blockade on control of growth of M. tuberculosis in during the first 24 hrs of whole blood culture. Symbols indicate median and IQR of 20 subjects. Divergent effects at peak levels approached statistical significance at 24 hrs (P=0.05 by repeated measures ANOVA), although none of the treatments differed significantly from untreated controls. Note: Overlapping error bars do not preclude differences by paired or repeated measures testing.

FIG. 3 depicts the effect of TNF blockade on apoptosis in 48 hr cultures of M. tuberculosis CF-stimulated monocytes. Symbols indicate median and IQR of 20 subjects. Apoptosis was measured by ELISA as histone associated cytoplasmic DNA. Apoptosis in cultures treated with peak therapeutic concentrations of infliximab was significantly greater than in control cultures by repeated measures ANOVA, but reached only one tenth the level reported in cultures with other pro-apoptotic stimuli.

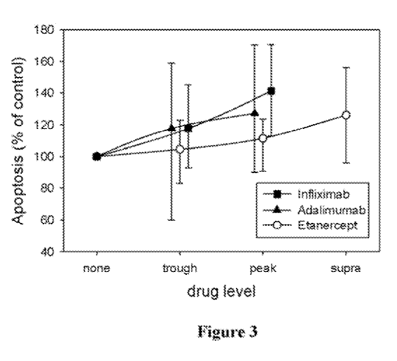

### SUMMARY OF THE INVENTION

The present invention is directed to cytokines and genes that are differentially affected by TNF blockers and the use of these genes and cytokines to help asses the TB risks of new immunosuppressive therapies, to help evaluate the effects of new TB vaccines, and to help assess TB susceptibility in persons exposed to Mycobacterium tuberculosis.

In certain embodiments, the present invention is directed to a method of analyzing the effect of a composition on the risk of TB infection comprising exposing the composition to a cytokine or gene associated with TB defense, wherein the cytokine or gene is capable of being deactivated by TNF blockade; and analyzing the results, wherein deactivation of the cytokine or gene indicates that the composition will have a higher probability of increasing the risk of TB infection compared to a composition that activates the cytokine or gene.

In certain embodiments, the present invention is directed to a method of analyzing the effect of a composition on the risk of TB infection comprising exposing the composition to a cytokine or gene associated with TB risk, wherein the cytokine or gene is capable of being deactivated by TNF blockade; and analyzing the results, wherein deactivation of the cytokine or gene indicates that the composition will have a higher probability of decreasing the risk of TB infection compared to a composition that activates the cytokine or gene.

### DETAILED DESCRIPTION

Differential regulation of cytokines and genes involved in protection from TB or in TB susceptibility may account for the differential TB risks of TNF blockers. The effects of these drugs (and another TNF antibody, adalimumab) on cytokines and genes activated by Mycobacterium tuberculosis in whole blood culture were studied. The profiles of two antibodies were very similar, but were quite different from the soluble receptor.

Blood cells and blood-derived dendritic cells were stimulated with M. tuberculosis culture filtrate or infected with M. tuberculosis. Supernatants were collected to examine cytokine expression. Cells were examined by flow cytometry for surface marker expression. Cellular mRNA was collected and examined by microarray. Infliximab and adalimumab, but not etanercept, inhibited antigen and mitogen-induced T cell activation and production of interferon gamma (IFNg), a cytokine known to be required for host defenses against TB. Infliximab and adalimumab inhibited production of granulocyte-monocyte colony stimulating factor (GM-CSF) to a greater extent than etanercept. GM-CSF is thought by some to have a contributing role in defenses against TB, but this is not certain. Etanercept inhibited production of transforming growth factor beta (TGFb), a regulatory cytokine that is associated with TB risk rather than protection. In contrast, infliximab had no effect, and adalimumab increased production of this cytokine.

381 genes were identified by microarray that were activated by M. tuberculosis culture filtrate. Of these, 113 were deactivated by infliximab and 109 by adalimumab. 89 genes were deactivated by both the TNF antibodies. In contrast, only 11 genes were deactivated by etanercept these 11 genes were also deactivated by both the antibodies. Approximately 60 of the 78 genes deactivated by both the TNF antibodies are annotated, meaning something is presently known regarding their function. These genes include GM-CSF, IFNg, superoxide dismutase, and several genes known to be activated by IFNg. One gene activated by TGFb was also identified; this gene was turned off by etanercept but not by adalimumab.

The experiments were conducted to reflect as accurately as possible conditions in vivo during therapeutic TNF blockade. Trough and peak drug concentrations were selected to reflect those during treatment. M. tuberculosis CF was selected based on its ability to stimulate TNF expression on monocytes as well as T cells (Wallis et al. Infect Immun 1993). Cultures were performed with fresh autologous plasma to permit complement activation by TNF-mAb immune complexes. Whole blood culture was used to permit expression of antibody-dependent cell-mediated cytotoxicity in mixed cell populations.

The main findings were that infliximab and adalimumab inhibited T cell activation and IFNγ production, whereas etanercept did not. IFNγ, like TNF, is essential for protection against tuberculosis. The inability to appropriately produce or respond to IFNγ strongly predisposes to tuberculosis, often resulting in disseminated infection even with attenuated mycobacteria (See Flynn et al. J Exp Med 1993; and Jouanguy et al. N Engl J Med 1996). Etanercept and infliximab differed in their effects on IFNγ production whether compared at equal or peak therapeutic concentrations. Adalimumab shared a profile similar to that of infliximab. These findings may indicate a fundamental relationship to drug mechanism of action rather than other factors. Although clinical experience with adalimumab is limited, recent post-marketing surveillance in Europe and North America reveals greater than anticipated rates of tuberculosis (See Schiff et al. Ann Rheum Dis 2006). These clinical and experimental findings together indicate adalimumab and infliximab may share similar risks of TB reactivation despite their differences in route of administration, dosing, and pharmacokinetics.

The mechanism of inhibition of IFNγ by infliximab and adalimumab otherwise remains uncertain. It could not be attributed to excess production of IL-10, which was inhibited equally by all three drugs at all tested concentrations. IL-10 production is increased in tuberculosis, in which it inhibits expression of IFNγ and costimulatory molecules (See Hirsch et al. J Infect Dis 1999; and Shrikant et al, 3 Immunol 1995). The findings indicate that inhibition of IL-10 by TNF blockade appears not to protect against reactivation of latent tuberculosis by infliximab. Other cytokines, such as transforming growth factor beta (TGFβ) and IFNα have also been implicated in regulation of IFNγ production in tuberculosis (See Manca et al. Proc Natl Acid Sci USA 2001; and Hirsch et al, Proc Natl Acad Sci USA 1996).

The TNF blockers had divergent effects on control of mycobacterial growth in whole blood culture. This intracellular infection model has been advocated as a tool to study new tuberculosis vaccines. Impaired control of mycobacterial growth in whole blood culture is evident in persons with HIV infection, or following depletion of CD4 or CD8 T cells, or addition of methylprednisolone or the TNF inhibitor pentoxifylline. Host effector mechanisms expressed during the first 24 hrs of whole blood culture may include innate bactericidal mechanisms of phagocytic cells, cellular cytotoxicity, and release of antibacterial peptides.

Induction of apoptosis in lamina propria T cells has been proposed as a therapeutic mechanism in Crohn's disease, as levels increase to normal following treatment with infliximab. Similar effects of infliximab and adalimumab on activated blood lymphocytes and monocytes of healthy donors have been observed by some but not all investigators. This variation may reflect methodologic differences, as levels of apoptosis appear to be greatest reports in which monocytes were cultured under low serum concentrations. The lack of significant apoptosis or necrosis in the present study indicates that IFNγ inhibition occurs in the absence of cell death.

The anti-TNF antibodies infliximab and adalimumab inhibited T cell activation and IFNγ production in vitro, whereas the soluble TNF receptor etanercept showed no effect. The risk of reactivation of latent M. tuberculosis infection posed by infliximab may reflect its ability both to block the effects of TNF and to inhibit production of IFNγ.

### EXAMPLE 1

### Materials and Methods

Subjects. Heparinized blood was collected from healthy volunteers. Experiments in which control of intracellular growth of M. tuberculosis or T cell responses to M. tuberculosis culture filtrate (CF) were assessed were conducted with tuberculin skin test positive donors. Other experiments were conducted without regard to skin test status.

Mycobacteria. M. tuberculosis H₃₇Rv culture filtrate was prepared as previously described in Wallis et al. Proc Natl Acad Sci USA 1990, M. tuberculosis H₃₇Ra was propagated in BACTEC 13A medium (Becton Dickinson, Sparks Md.) and frozen in aliquots, as described in Cheon et al. Clin Diagn Lab Immunol 2002. A standard curve relating inoculum size to days to positive (DTP) in BACTEC 12B was generated using inoculum volumes of 0.01 to 1000 μl. Cultures were scored as positive at a growth index (GI) of 30, using interpolation. The inoculum was selected as that volume calculated to become positive in 4.5 days.

Cytokine production was assessed in whole blood culture. Briefly, blood was diluted 1:4 with tissue culture medium (RPMI 1640 [Invitrogen, Carlsbad Calif.] with HEPES 25 mM). Cultures were stimulated with M. tuberculosis CF 5 μg/ml or phytohemagglutinin A (PHA, Sigma-Aldrich. St. Louis Mo.) 5 μg/ml. TNF blockers were added at trough and peak concentrations reached in blood during therapy, based on information provided by the manufacturer or published studies (etanercept, 1 and 2 μg/ml; infliximab, 5 and 80 μg/ml; and adalimumab, 5 and 10 μg/ml). Etanercept was also tested at a supratherapeutic concentration of 5 μg/ml. Blood was cultured in 24 well culture plates at 37° C. in 5% CO₂. Supernatants were collected after 5 days and stored at −70° C. IFNγ and IL-10 were analyzed by ELISA (R&D Systems, Minneapolis Minn.) according to manufacturer's instructions.

Control of intracellular M. tuberculosis growth was assessed in whole blood culture. Briefly, blood was mixed 1:1 with tissue culture medium in 2 ml vials and was inoculated with M. tuberculosis H₃₇Ra. TNF blockers were added at the concentrations indicated. The vials were sealed and incubated at 37° C. with slow constant mixing. After 24 or 96 hrs, blood cells were pelleted by centrifugation, and the supernatant discarded. The pelleted cells were lysed, and recovered bacilli inoculated into BACTEC 12B medium. Growth indices were monitored daily. The extent of bacillary growth or killing was determined by comparing DTP values of the completed cultures with the inoculum. Data management and calculation of bacillary survival was performed using software written by one of the authors (RSW); it is available by request to the author.

T cell activation, apoptosis and necrosis. Heparinized blood 600 μl was mixed with an equal volume of tissue culture medium in polypropylene tubes, and stimulated with PHA 5 μg/ml or M. tuberculosis CF 5 μg/ml. TNF blockers were added at the concentrations indicated. Cultures were incubated at 3° C. for 24 or 72 hrs. RBC were lysed with PharM Lyse (BD Biosciences, Sparks Md.). Cells were labeled using the following monoclonal antibodies: CD4-PE, CD8-PE CD69-APC, annexin V-FITC, 7AAD, and isotype mouse IgG_{l,k}-APC (BD), according to the manufacturer's instructions. Labeled cells were detected by flow cytometry (FACSCalibur, BD). Mononuclear cells were selected based on forward and side light scattering. A minimum of 100,000 events were counted for each experiment.

Monocyte apoptosis and necrosis. Mononuclear cells were isolated by density gradient sedimentation over Ficoll-Paque PLUS (Amersham Biosciences AB, Uppsala, Sweden). Cells were cultured at 37° C. in 5% CO₂ at a density of 10⁵/ml in 24 well plates pre-coated with 50 μl of equal volumes of heat inactivated fetal bovine serum (Invitrogen) and pooled human serum (Gemini BioProducts, Woodland Calif.). Nonadherent cells were removed after 90 minutes by repeated washing with warmed medium. Remaining adherent cells were cultured in a 1:1 mixture of autologous plasma plus medium. All cultures were stimulated with M. tuberculosis CF 5 μg/ml. TNF blockers were added at the concentrations indicated. Cultures were incubated for 24 or 48 hrs. Apoptosis was determined by cytoplasmic histone-associated DNA fragments using the Cell Death Detection ELISA^{PLUS} (Roche Applied Sciences, Indianapolis Ind.) according to the manufacturer's instructions. Briefly, cells were sedimented at 200 g for 10 minutes in the wells in which they had been cultured. The supernatant was removed and discarded. Cells were then lysed by the addition of 200 μl lysis buffer. After 30 minutes incubation at room temperature, the culture plate was centrifuged at 200 g for 10 min. Twenty μl of the supernatant was transferred to a streptavidin-coated microplate. Histone-associated DNA was then detected by sandwich ELISA. Results were expressed as the mean of 4 replicate wells. The change in apoptosis was calculated as the ratio of the optical densities of treated vs. control cultures, less the background optical density.

Statistical analysis. The Friedman Repeated Measures Analysis of Variance (RM ANOVA) on Ranks was used to determine whether the overall differences in median values among the treatments were greater than could be expected by chance. If significant differences were found, multiple post-hoc comparisons were performed using Dunn's method to determine which treatments differed significantly from control. This approach emphasizes differences among treatments within individuals, thus increasing the power of the analysis. It also minimizes the risk of a type 2 error due to repeated comparisons. All statistical analyses were performed using SigmaStat (Systat Software, Point Richmond Calif.).

### Results

Antigen and mitogen-induced T cell activation. The effects of TNF blockade on T cell expression of CD69 (an early marker of activation) were studied in antigen M. tuberculosis culture filtrate, CF) and mitogen (PHA)-stimulated whole blood cultures of 24 hrs duration. Resting cells failed to show detectable CD69 expression (not shown). As indicated in Table 1, both adalimumab and infliximab inhibited T cell activation. The greatest effect was seen with infliximab, which reduced antigen-induced CD4 activation to 30% of baseline. In contrast, etanercept caused no significant effect.

TABLE 1
Effects of TNF blockade on T cell activation (CD69 expression).
Values indicate medians of 10 subjects.

| | Stimulus | | |
|---|---|---|---|
| | TB CF | PHA | PHA |
| | Cell type | | |
| | CD4 | CD4 | CD8 |
| | % CD69+ cells | | |
| No treatment | 0.73 | 14.4 | 11.1 |
| Etanercept 2 μg/ml | 0.60 | 15.1 | 9.1 |
| Adalimumab 5 | 0.37* | 10.5 | 7.8 |
| μg/ml | | | |
| Infliximab 80 μg/ml | 0.22* | 10.1* | 5.9 |
| P | .003 | .015 | .064 |

Asterisks indicate significance compared to untreated controls by repeated measures ANOVA on ranks, followed by post-hoc testing by Dunn's method. Adalimumab and infliximab both significantly inhibited antigen-induced CD4 activation. Similar effects of smaller magnitude were also observed in mitogen-stimulated cells. Etanercept had no significant effect.

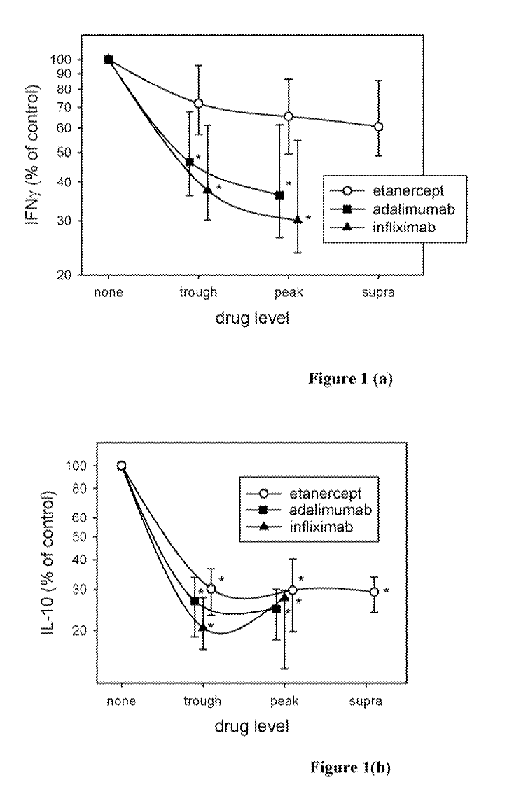

Antigen-induced IFNγ and IL-10 expression. The effects of TNF blockade on M. tuberculosis CF-induced IFNγ and IL-10 production were examined in whole blood cultures of 5 days duration. Median IFNγ production without TNF blockade was 1082 pg/ml (interquartile range [IQR], 651-1621). Infliximab and adalimumab suppressed IFNγ production to 30% of baseline (P<0.05, FIG. 1 left panel). Significant effects were observed even at trough drug concentrations (5 μg/ml), whether compared to control cultures without drug or to corresponding cultures with etanercept. In contrast, etanercept showed no statistically significant effect on IFNγ compared to control cultures, even when tested at a supratherapeutic concentration (5 μg/ml). To ensure that these findings were not due to a non-specific effect of antibody, an additional experiment was performed in 6 subjects by adding human immunoglobulin for intravenous administration (IVIG, Gamunex) to antigen-stimulated cultures. Addition of IVIG 80 μg/ml tended to increase IFNγ increase IFNγ production by 12% (1161 pg/ml vs. 1097 pg/ml in paired control cultures, P=0.065), an effect that could not account for the inhibition of IFNγ we observed with TNF mAbs.

Median IL-10 production by cells stimulated with M. tuberculosis CF was 875 pg/ml (IQR, 399-1295). Infliximab, adalimumab, and etanercept all suppressed IL-10 production to 20-30% of baseline values, regardless of drug concentration (FIG. 1, right panel).

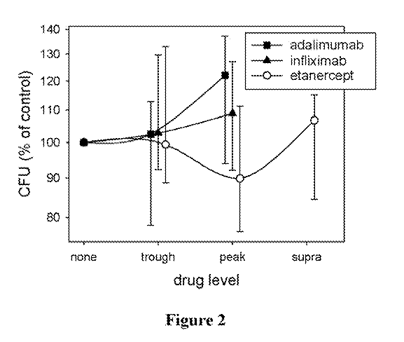

Control of intracellular M. tuberculosis growth. The effect of TNF blockade on control of intracellular mycobacterial growth was assessed in whole blood cultures infected with M. tuberculosis H37Ra. Mycobacterial viability declined by 70% (from 1100 to 344 CFU) during the first 24 hrs, and did not change further during hours 24 to 96, consistent with prior studies using this model. Adalimumab and etanercept had divergent, concentration-dependent effects on control of intracellular growth during the first 24 hrs (FIG. 2). This difference approached statistical significance at peak drug levels (P=0.05). Mycobacterial viability was not further affected by TNF blockade turning the subsequent 72 hrs (not shown).

T cell apoptosis and necrosis. The effects of TNF blockade on T cell apoptosis and necrosis were studied in antigen and mitogen-stimulated whole blood culture of 24 and 72 hrs duration (table 2). Induction of apoptosis was not observed under any of the conditions tested. Indeed, the only significant effect observed was inhibition of apoptosis by all three TNF blockers in PHA-stimulated cultures. Other researchers have reported that induction of apoptosis by infliximab occurs only in activated T cells. Since we had observed inhibition of T cell activation by TNF blockade (Table 1), we therefore repeated the analysis in PHA-stimulated cultures after gating on activated (CD69+) cells. Higher levels of apoptosis and necrosis were indeed observed this cell population; however, no effects of TNF blockade were identified.

TABLE 2

| Stimulus: | TB CF | PHA | PHA | PHA | PHA | PHA | PHA | PHA | PHA |
|---|---|---|---|---|---|---|---|---|---|
| Cell type: | CD4 | CD4 | CD4 | CD8 | CD8 | CD4 | CD4 | CD8 | CD8 |
| Culture duration: | 24 hr | 24 hr | 72 hr | 24 hr | 72 hr | 24 hr | 72 hr | 24 hr | 72 hr |
| Gating: | All cells | | | | | CD69+ cells | | | |
| | % Annexin V+ 7AAD− | | | | | | | | |
| | % 7AAD+ | | | | | | | | |
| No treatment | 2.9 | 7.7 | 13 | 13 | 21 | 10 | 23 | 16 | 34 |
| | 1.2 | 2.0 | 20 | 3.2 | 19 | 2.9 | 13 | 1.9 | 11 |
| Etanercept 2 μg/ml | 3.2 | 4.7* | 15 | 12 | 21 | 7.9 | 25 | 18 | 37 |
| | 1.1 | 1.8 | 21 | 2.1 | 17 | 2.4 | 12 | 2.3 | 9.8 |
| Adalimumab 5 μg/ml | 3.0 | 5.8* | 14 | 12 | 18 | 8.5 | 24 | 17 | 35 |
| | 1.3 | 2.0 | 24 | 2.4 | 19 | 2.4 | 14 | 1.7 | 11 |
| Infliximab 80 μg/ml | 2.5 | 6.0* | 13 | 11 | 18 | 8.2 | 22 | 15 | 33 |
| | 0.9 | 1.5 | 23 | 2.7 | 21 | 2.6 | 14 | 2.0 | 13 |
| P | .22 | .008 | .51 | .28 | .16 | .13 | .65 | .20 | .90 |
| | .16 | .06 | .90 | .33 | .68 | .16 | 43 | 83 | 43 |

Effect of TNF blockade on apoptosis (annexin V+ 7AAD−, upper values) and necrosis (7AAD+, lower values) in antigen and mitogen-stimulated T cells. Values indicate median percent positive cells of a minimum of 9 subjects. Asterisks indicate significance compared to untreated controls by repeated measures ANOVA on ranks. Apoptosis was inhibited by TNF blockade in 24 hr cultures of unselected PHA-stimulated CD4 cells, but this effect was abolished when analysis was restricted to activated (CD69+) cells. Apoptosis was not induced by TNF blockade under any conditions tested.

Monocyte apoptosis and necrosis. The effects of TNF blockade on apoptosis in monocytes activated with M. tuberculosis CF were studied after 24 and 48 hrs incubation, by detection by ELISA of histone-associated cytoplasmic DNA. This method permitted the analysis of drug effects without requiring the removal of cell monolayers by mechanical scraping or enzymatic digestion it also avoided monocyte loss due to adherence or clumping in activated mixed cell suspensions. No significant effects were observed in 24 hr cultures not shown). At 48 hrs, peak concentrations of infliximab resulted in a 40% increase in histone-associated DNA compared to TB-stimulated control cultures, whereas neither etanercept nor adalimumab caused a significant effect (P<0.05 compared to control, FIG. 4). However, the biological significance of this observation is uncertain, as other apoptotic stimuli (e.g., serum starvation) produce up to 10-fold greater effects using this method.

Necrosis was assessed in the treated monolayers as trypan blue permeability. Fewer than 3% of cells were trypan blue positive, regardless of anti-TNF treatment.

### EXAMPLE 2

Whole blood cultures of 6 healthy TB skin test positive donors. 791 genes were identified as significantly up or down regulated by M. tuberculosis (³1.5 fold change and P<05 after adjustment for multiple testing). Effects of TNF blockers on these genes were then analyzed. 75 genes of the 791 genes were deactivated by infliximab, 60 by adalimumab, and 40 by etanercept. Analysis by Venn diagram shows 3 nearly concentric circles, with the set of 40 etanercept-deactivated genes contained entirely within those of the two antibodies. Analysis by gene ontology of the recognized biologic processes and molecular functions affected by infliximab and adalimumab but not by etanercept reveals differential effects on chemokines and apoptosis, and may indicate effector memory T cells as the locus of these effects. This study may also reveal possible roles in defenses against mycobacterial infection for some genes that are currently with annotations.

TABLE 3
Genes activated (or deactivated) by M. tuberculosis in whole
blood culture that are subsequently deactivated (or activated) by
TNF blockers:

| INFL | ADAL | ETAN |
|---|---|---|
| Hs.104624 | Hs.104624 | Hs.112341 |
| Hs.104879 | Hs.112341 | Hs.143929 |
| Hs.112341 | Hs.11638 | Hs.148741 |
| Hs.11638 | Hs.118400 | Hs.1547 |
| Hs.118400 | Hs.143929 | Hs.1722 |
| Hs.126256 | Hs.148741 | Hs.179838 |
| Hs.148741 | Hs.1547 | Hs.204238 |
| Hs.1547 | Hs.1722 | Hs.2233 |
| Hs.15725 | Hs.179838 | Hs.224012 |
| Hs.159430 | Hs.204238 | Hs.236516 |
| Hs.1722 | Hs.211238 | Hs.238964 |
| Hs.179838 | Hs.2233 | Hs.241570 |
| Hs.196384 | Hs.224012 | Hs.25362 |
| Hs.204238 | Hs.236516 | Hs.306199 |
| Hs.2233 | Hs.238964 | Hs.378150 |
| Hs.224012 | Hs.25362 | Hs.382202 |
| Hs.236516 | Hs.288034 | Hs.437638 |
| Hs.238964 | Hs.306199 | Hs.440544 |
| Hs.241570 | Hs.319171 | Hs.449207 |
| Hs.25362 | Hs.378150 | Hs.471991 |
| Hs.288034 | Hs.382202 | Hs.480653 |
| Hs.306199 | Hs.390736 | Hs.487046 |
| Hs.319171 | Hs.437322 | Hs.502328 |
| Hs.382202 | Hs.437638 | Hs.513147 |
| Hs.390736 | Hs.440544 | Hs.514107 |
| Hs.437322 | Hs.449207 | Hs.517265 |
| Hs.437638 | Hs.459106 | Hs.519909 |
| Hs.440544 | Hs.468840 | Hs.520819 |
| Hs.444082 | Hs.471991 | Hs.525634 |
| Hs.449207 | Hs.480653 | Hs.567311 |
| Hs.459106 | Hs.487046 | Hs.571296 |
| Hs.468840 | Hs.488173 | Hs.593168 |
| Hs.471991 | Hs.492208 | Hs.597550 |
| Hs.480653 | Hs.502328 | Hs.646274 |
| Hs.48384 | Hs.502875 | Hs.695989 |
| Hs.487046 | Hs.505874 | Hs.75703 |
| Hs.488173 | Hs.514107 | Hs.81134 |
| Hs.492208 | Hs.517265 | Hs.81328 |
| Hs.502328 | Hs.518662 | Hs.8162 |
| Hs.502875 | Hs.519909 | Hs.86724 |
| Hs.505874 | Hs.520819 | |
| Hs.513147 | Hs.525634 | |
| Hs.514107 | Hs.546392 | |
| Hs.515351 | Hs.567311 | |
| Hs.517265 | Hs.571296 | |
| Hs.518662 | Hs.591463 | |
| Hs.519909 | Hs.593168 | |
| Hs.520819 | Hs.597550 | |
| Hs.525634 | Hs.62661 | |
| Hs.529609 | Hs.646274 | |
| Hs.531251 | Hs.658407 | |
| Hs.535713 | Hs.665541 | |
| Hs.546392 | Hs.6790 | |
| Hs.567311 | Hs.695989 | |
| Hs.571296 | Hs.696045 | |
| Hs.591338 | Hs.75703 | |
| Hs.593168 | Hs.81134 | |
| Hs.597550 | Hs.81328 | |
| Hs.62661 | Hs.8162 | |
| Hs.643447 | Hs.86724 | |
| Hs.646274 | | |
| Hs.654558 | | |
| Hs.656476 | | |
| Hs.658407 | | |
| Hs.6790 | | |
| Hs.695989 | | |
| Hs.696045 | | |
| Hs.696064 | | |
| Hs.75498 | | |
| Hs.75703 | | |
| Hs.81134 | | |
| Hs.81328 | | |
| Hs.8162 | | |
| Hs.86724 | | |
| Hs.99962 | | |

What is claimed:

1. A method of analyzing the effect of a composition on the risk of TB infection comprising:
i. exposing the composition to a cytokine or gene associated with TB defense, wherein the cytokine or gene is capable of being deactivated by INF blockade; and
ii. analyzing the results, wherein deactivation of the cytokine or gene indicates that the composition will have a higher probability of increasing the risk of TB infection compared to a composition that activates the cytokine or gene.

2. A method of analyzing the effect of a composition on the risk of TB infection comprising:
i. exposing the composition to a cytokine or gene associated with TB risk, wherein the cytokine or gene is capable of being deactivated by TNF blockade; and
ii. analyzing the results, wherein deactivation of the cytokine or gene indicates that the composition will have a higher probability of decreasing the risk of TB infection compared to a composition that activates the cytokine or gene.

3. The method of claim 1, wherein the cytokine is IFNγ and the gene is GM-CSF or superoxide dismutase.

4. The method of claim 2, wherein the cytokine is TGFb.

5. A model for the assessment of TB susceptibility in a subject exposed to M. Tuberculosis comprising testing the affect of a composition on cytokines or genes that are differentially affected by TNF blockade.
